(19) United States
(12) Patent Application Publication
(10) Pub. No.: US 2024/0139039 A1
(43) Pub. Date: May 2, 2024

(54) REUSABLE ABSORBENT CARRIER INSERT AND DISPOSABLE INSERT COMBINATIONS, ASSEMBLIES, AND METHODS OF ASSEMBLY

(71) Applicant: The Procter & Gamble Company, Cincinnati, OH (US)

(72) Inventors: Yin CHEN, Königstein (DE); Udo Friedel SCHOENBORN, Bad Soden (DE)

(21) Appl. No.: 18/496,984

(22) Filed: Oct. 30, 2023

Related U.S. Application Data
(60) Provisional application No. 63/420,745, filed on Oct. 31, 2022.

(51) Int. Cl. A61F 13/505; A61F 13/56

(52) U.S. Cl. CPC A61F 13/505; A61F 13/5605

(57) ABSTRACT

Embodiments relate to combinations, assemblies, and methods of assembly of a reusable absorbent carrier insert and disposable insert combination comprising a reusable absorbent carrier insert and a disposable insert. The reusable absorbent carrier insert comprises a carrier portion and has a first capacity-to-caliper ratio. The disposable insert comprises an absorbent material and has a second capacity-to-caliper ratio. The disposable insert is configured to be received and contained within the carrier portion of the reusable absorbent carrier insert to form the reusable absorbent carrier insert and disposable insert combination. The first length is greater than the second length, the first width is greater than the second width, and the first capacity-to-caliper ratio is less than the second capacity-to-caliper ratio.

### CROSS REFERENCE TO RELATED APPLICATION

This application claims the benefit of U.S. Provisional Application No. 63/420,745, filed Oct. 31, 2022, the substance of which is incorporated herein by reference.

### TECHNICAL FIELD

The present disclosure generally relates to reusable absorbent carrier insert and disposable insert combinations and absorbent assemblies including the same, and in particular to systems and methods for forming the reusable combinations and assemblies.

### BACKGROUND

Infants and other incontinent individuals wear absorbent articles and assemblies such as diapers or other absorbent pants to receive and contain urine and other body fluids such as a feces. Such absorbent articles may include reusable absorbent inserts. However, while the reusable absorbent inserts may be cleaned for reuse, they may take an extended period of time to dry after such cleaning. Conventional reusable absorbent inserts may have a disadvantage in capacity and dryness versus disposable articles that may use super absorbent polymers. Such a disadvantage may be compensated with multiple layers of fabrics which make the insert thicker, and hence appears bulkier and less fitted when put on baby together with a diaper outer cover, and also takes an extended period of time to dry after laundry.

Based on the foregoing, there is a need for alternative absorbent inserts and assemblies, and methods of use and manufacture, to more efficiently absorb bodily fluids and allow for decreased drying times after cleaning as well as an improved capacity and dryness and improved fit.

### SUMMARY

According to the subject matter of the present disclosure, a reusable absorbent carrier insert and disposable insert combination comprises a reusable absorbent carrier insert and a disposable insert. The reusable absorbent carrier insert comprises a carrier portion, a first length measured along a central longitudinal axis of the reusable absorbent carrier insert, and a first width measured along a central lateral axis of the reusable absorbent carrier insert. The reusable absorbent carrier insert has a first capacity-to-caliper ratio. The disposable insert comprises an absorbent material, a second length measured along a central longitudinal axis of the disposable insert, and a second width measured along a central lateral axis of the disposable insert. The disposable insert has a second capacity-to-caliper ratio. The reusable absorbent carrier insert is configured to receive and retain the disposable insert. The disposable insert is configured to be received and contained within the carrier portion of the reusable absorbent carrier insert to form the reusable absorbent carrier insert and disposable insert combination. The first length is greater than the second length, the first width is greater than the second width, and the first capacity-to-caliper ratio is less than the second capacity-to-caliper ratio.

In another embodiment, an absorbent pant comprises an outer shell comprising an interior pocket, an interior portion, or combinations thereof, and a reusable absorbent carrier insert and disposable insert combination configured to be received at least in one of in the interior pocket of the outer shell of the absorbent pant, on top of the interior portion of the outer shell, or combinations thereof. The reusable absorbent carrier insert and disposable insert combination comprises a reusable absorbent carrier insert and a disposable insert. The reusable absorbent carrier insert comprises a carrier portion, a first length measured along a central longitudinal axis of the reusable absorbent carrier insert, and a first width measured along a central lateral axis of the reusable absorbent carrier insert, wherein the reusable absorbent carrier insert has a first capacity-to-caliper ratio. The disposable insert comprises an absorbent material, a second length measured along a central longitudinal axis of the disposable insert, and a second width measured along a central lateral axis of the disposable insert, wherein the disposable insert has a second capacity-to-caliper ratio. The reusable absorbent carrier insert is configured to receive and retain the disposable insert. The disposable insert is configured to be received and contained within the carrier portion of the reusable absorbent carrier insert to form the reusable absorbent carrier insert and disposable insert combination. The first length is greater than the second length, the first width is greater than the second width, and the first capacity-to-caliper ratio is less than the second capacity-to-caliper ratio.

In yet another embodiment, a method of forming a reusable absorbent carrier insert and disposable insert combination comprises receiving a disposable insert comprising an absorbent material within a carrier portion of a reusable absorbent carrier insert and containing the disposable insert in the carrier portion in a self-locking configuration upon receipt. The reusable absorbent carrier insert has a first capacity-to-caliper ratio, the disposable insert has a second capacity-to-caliper ratio, and the first capacity-to-caliper ratio is less than the second capacity-to-caliper ratio.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is an interior side view of an absorbent assembly, showing an interior portion that faces toward a wearer, according to one or more embodiments shown and described herein;

FIG. 2 is an exterior side view of the absorbent assembly of FIG. 1, showing an exterior portion that faces away from the wearer;

FIG. 3 is an interior side view of the absorbent assembly of FIG. 1, showing a reusable absorbent carrier insert partially disposed in an interior pocket defined at an end of an outer shell of the absorbent assembly;

FIG. 4 is an interior side view of another absorbent assembly, showing a reusable absorbent carrier insert partially disposed in an interior pocket defined at a side edge of an outer shell of the absorbent assembly, according to one or more embodiments shown and described herein;

FIG. 5A is a schematic view of a reusable absorbent carrier insert and disposable insert combination including a belt with two open ends, according to one or more embodiments shown and described herein;

FIG. 5B is a schematic view of a reusable absorbent carrier insert and disposable insert combination including a pair of belts with two open ends, according to one or more embodiments shown and described herein;

FIG. 6A is a schematic view of a reusable absorbent carrier insert and disposable insert combination including a pouch with a partially extending longitudinal incision, according to one or more embodiments shown and described herein;

FIG. 6B is a schematic view of a reusable absorbent carrier insert and disposable insert combination including a pouch with a fully extending longitudinal incision, according to one or more embodiments shown and described herein;

FIG. 7 is a schematic view of a reusable absorbent carrier insert and disposable insert combination including a belt with two open ends and a pouch with a partially extending longitudinal incision, according to one or more embodiments shown and described herein;

FIG. 8 is a schematic view of a reusable absorbent carrier insert and disposable insert combination including a pouch with a pair of flaps, according to one or more embodiments shown and described herein;

FIG. 9 is a schematic view of a reusable absorbent carrier insert and disposable insert combination including a pouch with a diagonal incision, according to one or more embodiments shown and described herein; and

FIG. 10 is a schematic view of a reusable absorbent carrier insert and disposable insert combination including a pouch with a partially extending longitudinal incision that is configured to be folded over the disposable insert, according to one or more embodiments shown and described herein.

### DETAILED DESCRIPTION

Reference will now be made in detail to embodiments of reusable absorbent carrier insert and disposable insert combinations and assemblies, examples of which are illustrated in the accompanying drawings. Embodiments herein are directed to alternative absorbent insert combinations and assemblies and methods of use and manufacture to more efficiently absorb bodily fluids and allow for decreased drying times after cleaning. Whenever possible, the same reference numerals will be used throughout the drawings to refer to the same or like parts. Various embodiments of such absorbent patents, systems, and methods will be described in further detail herein with specific reference to the appended drawings.

The reusable absorbent carrier inserts may be made of various foldable and non-foldable configurations and may be made of textile materials or fabrics, fleece, terry, microterry, cotton, bamboo based, Lyocell, or combinations thereof. The materials may be made of synthetic yarns or yarns of natural origin, including but not limited to PET, PLA, cotton, bamboo, hemp, Lyocell. Further, the materials may be of any fabric construction, including but not limited to knitted, woven, or non-woven. Durable, multiple layers may be fastened together such as through an overlock stitch, tape, glue, mechanical bonding, or like fastening items. The layers of the reusable absorbent carrier inserts may be made of textile materials or fabrics such as terry, microterry, cotton, bamboo based, Lyocell, or combinations thereof. In embodiments, the reusable absorbent carrier inserts may be made of one or more fabric types, such as without limitation types provided through weaving, knitting, crocheting, felting, or other nonwovens types. Some executions of fabric types via weaving, knitting, warp-knitting may include, and not be limited to, terry-cloth, waffle fabric, jacquard, jersey, 3D fabrics, and the like. Material types may include without limitation (1) natural fibers such as cotton, hemp, silk, wool, mineral, and the like; (2) man-made fibers such as polyester, polypropylene, re-constituted cellulose (such as viscose and/or Lyocell), and the like, or (3) combinations thereof, and/or may include (4) specialty fibers such as micro fibers, hollow fibers, and the like.

“Absorbent article” of the absorbent assemblies as described herein means a device that absorbs and contains body exudates and, more specifically, devices that are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like. Nonlimiting examples of absorbent articles are disclosed in U.S. Pat. Nos. 8,998,870, 9,089,456, 8,435,223, 9,011,402, 8,808,263, 8,759,605 and 8,932,273 and 9,078,789.

“Absorbent insert” and “insert” and “reusable absorbent (carrier) insert” mean a component of a wearable absorbent article that is adapted to contain and/or absorb urine, feces, menses or any combination thereof, and is adapted to be installable and removable as a modular unit, from an outer shell of an absorbent assemble as described herein.

“Outer shell” or “outer cover” means a component of a wearable absorbent article as an absorbent assembly that is adapted to be worn about the lower torso of a wearer, and is adapted to support an absorbent insert and hold the insert close to the wearer's body. In various embodiments, the outer cover is reusable.

“Reusable,” when referring to a component means that the component is adapted to be used for its intended purpose after initial use without substantial destruction of any portions of the component necessary for as-new functionality. For example, a reusable outer cover means an outer cover that is adapted to permit removal of at least a first insert, and replacement thereof with at least a second insert, without substantial destruction of any portions of the outer cover that are necessary to provide the substantial as-new functionality of the outer cover, and without the necessity of any repair or reconstruction following such insert replacement.

“Disposable,” in reference to absorbent (which may be superabsorbent) articles, means that the absorbent articles, are generally not intended to be laundered or otherwise restored or reused as absorbent articles (i.e., they are intended to be discarded after a single use and, preferably, to be recycled, composted or otherwise discarded in an environmentally compatible manner). Disposable absorbent articles often comprise adhesive between the layers and/or elements to hold the article together (e.g., ear panels, side panels, and belts are joined to the chassis via adhesive and the layers of the ear panels, side panels, belts, and chassis are joined together using adhesive). Alternatively, heat and/or pressure bonding are used with the adhesive or in place of the adhesive. In such instances portions of the material layers may become partially melted and pressed together such that once cooled they are physically bonded together. Nonwovens (including, for example, polypropylene, polyethylene, etc.) adhesives (including, for example, styrenic block copolymers (e.g., SIS, SBS)), and absorbent gelling material (AGM) make up more than 50%, more than 75%, and often more than 90% of the disposable absorbent article weight. And, a core comprising the AGM is often held within the chassis in a manner that would encapsulate and contain the AGM under normal conditions. Such disposable (super)absorbent articles typically have an absorbent capacity of greater than about 100 mL of fluid and can have capacities of up to about 500 mL of fluid or more. Stitching (including the use of thread) and/or woven materials are typically not used to make a disposable absorbent article. If stitching or woven materials are used, they make up an extremely small percentage of the disposable absorbent article. Some landing zones of disposable absorbent articles for fasteners can comprise a woven material, but no other part of a disposable absorbent article typically comprises woven materials. The absorbent material may comprise superabsorbent polymers, a mixture of superabsorbent polymers and air felt, only air felt, and/or a foam. In some instances, the absorbent material may comprise at least 80%, at least 85%, at least 90%, at least 95%, at least 99%, or up to 100% superabsorbent polymers, by weight of the absorbent material. In such instances, the absorbent material may free of air felt, or at least mostly free of air felt—in such cases the superabsorbent material may comprise AGM that may be held in place by an adhesive, such as a thermoplastic adhesive.

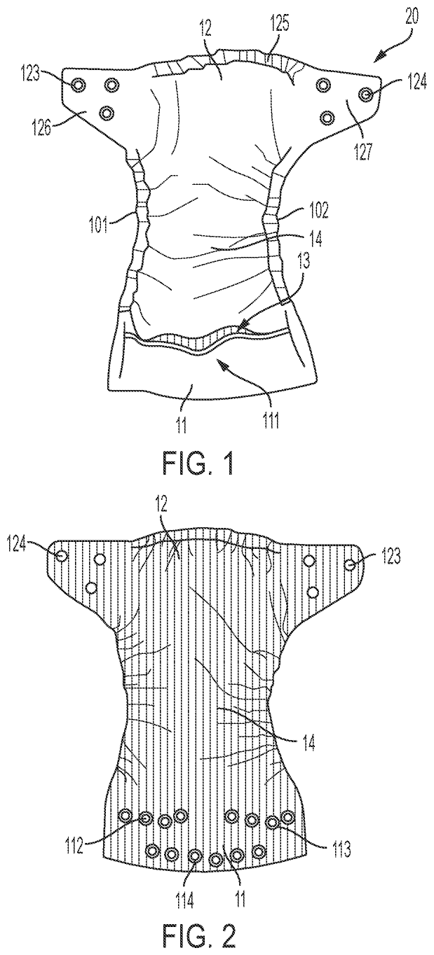

In aspects, and referring to FIGS. 1-4, an absorbent assembly 20 includes an outer shell 21 having a front portion 11 and a back portion 12 connected by an intermediate portion 14 forming a crotch region between a front waist region defined by the front portion 11 and a back waist region defined by the back portion 12. The outer shell 21 may include intermediate elastic strips 101 and 102 attached to the inner panel of FIG. 1 and outer panel of FIG. 2. Nonlimiting examples of outer shells are disclosed in U.S. Pat. Nos. 9,387,138 and 8,435,223.

The outer shell 21 and/or layers or portions thereof may be made of any durable or semi-durable knitted, woven or nonwoven textile or textile-like material that is appropriately compatible with skin of the intended wearer(s) and may include elastomeric materials. In embodiments, the reusable absorbent carrier inserts 15, 245 described herein and/or layers or portions thereof may be made of any durable or semi-durable knitted, woven or nonwoven textile or textile-like material that is appropriately compatible with skin of the intended wearer(s) and may include elastomeric material. The outer shell 21 may be formed of a single layer of a durable or semi-durable material, or may have two or more layers in the front region and/or rear region. Suitable examples are described in U.S. application Ser. Nos. 12/687,493; 12/687,412; 12/687,528; and Ser. No. 12/687,425 (all by Roe et al.). Non-limiting examples of fibers, nonwovens and laminates of nonwovens and films that might be considered for use as semi-durable outer cover materials may be found in U.S. Pat. Nos. 7,223,818; 7,211,531; 7,060,149; 6,964,720; 6,905,987; 6,890,872; 6,884,494; 6,878,647; and 5,518,801; and U.S. Published Applications Nos. 2008/0319407; 2008/0045917; 2007/0293111; 2007/0287983; 2007/0287348; 2007/0249254; 2007/0203301; and 2005/0164587. In some embodiments, the surfaces of both the interior, wearer-facing and exterior, garment-facing sides of the outer shell 21 may be formed from the same materials. However, due to different functional needs of the garment-facing surface and the wearer-facing surface, it may be desirable that the exterior surfaces comprise different materials.

In nonlimiting examples, the wearer-facing surface of the outer shell 21 may be formed from a first material or first combination of materials (collectively referred to as “first material” hereinafter). The material selected may include hydrophilic fibers, or fibers treated to be hydrophilic. This may be desired in some circumstances to cause the material forming wearer-facing surface to more readily absorb liquid, or transmit liquid therethrough. This may serve to provide supplemental absorbency within the outer shell 21 for an event in which liquid exudates escape the insert, reducing the likelihood that the outer shell 21 will leak. Alternatively, it may provide one way of communicating to the user that liquid exudates have escaped the insert, by causing wetness to be transmitted through to the outer cover such that wetness is sensed by the wearer and/or visible on exterior surfaces. Alternatively, it may serve to provide a layer that tends to draw moisture away from the skin, for a drier, more comfortable feel. Additionally, or alternatively, it may be desirable to form the wearer-facing surface from an apertured material to facilitate transfer of exudates through the wear-facing surface and onto the absorbent insert. In some nonlimiting examples, the material may include fibers having hydrophobic properties, providing enhanced liquid containment attributes to the wearer-facing layer.

The garment-facing surface of the outer cover may be formed by a second material, or a second combination of materials (collectively referred to as “second material” hereinafter). The material selected may include fibers having hydrophobic properties, providing enhanced liquid containment attributes to such layer or may include a liquid impermeable material. In another example, however, it may be desirable in some circumstances for the selected material to include hydrophilic fibers, or fibers treated to be hydrophilic, so as will cause the material to more readily absorb liquid. This may serve to provide supplemental absorbency within the outer cover for the event in which liquid exudates escape the insert, or to provide one way of communicating to the user that liquid exudates have escaped the insert. Additionally, in some circumstances, it may be desirable that the material selected have soft tactile properties so as to have a pleasant feel that the user and/or wearer find attractive. The material also may be selected so as to have a desired appearance, including but not limited to coloration, sheen, and/or texture.

The first material and the second material may differ by one of the group consisting of: texture, caliper, elasticity, opacity, water resistance, absorbency, breathability and combinations thereof.

In nonlimiting examples, the outer shell 21 may be reversible such that both the surfaces are adapted to function as the wearer-facing surface and the garment-facing surface. This may provide, for example, variation in visible graphics or colors, textures and/or breathability against skin, liquid wicking properties and the like for the wearer. Layers or other elements of the outer shell 21 may be joined to each other via any suitable mechanism, including, for example, adhesives, mechanical bonding, ultrasonic bonding, sewing, stitching, serging, edging, and the like.

In nonlimiting examples, an elasticized waist feature of the outer shell 21 comprises elastic strands or ribbons joined to the nonwoven and/or textile layer(s). In further nonlimiting examples, the elasticized waist feature comprises a laminate of one or more textile and/or nonwoven layers and one or more elastomeric films. The elasticized waist element may comprise one or more rugosities if the elastic material is strained prior to lamination. In other nonlimiting examples, the layers of the elastic laminate may be joined at zero applied strain and subsequently activated. In alternative embodiments, the waist feature may be inelastic. In such configurations, the waist feature may provide additional anchoring about the waist of the wearer. In some embodiments, elastic elements are affixed within the outer shell 21 only at or near one or both of their respective ends, e.g., within a pouch, tube or envelope structure formed of outer cover material—referred to herein as a “drawstring elastic”. The outer shell 21 may also include anchoring supplements, bands or systems thereof as described in more detail in U.S. Pat. No. 8,932,273.

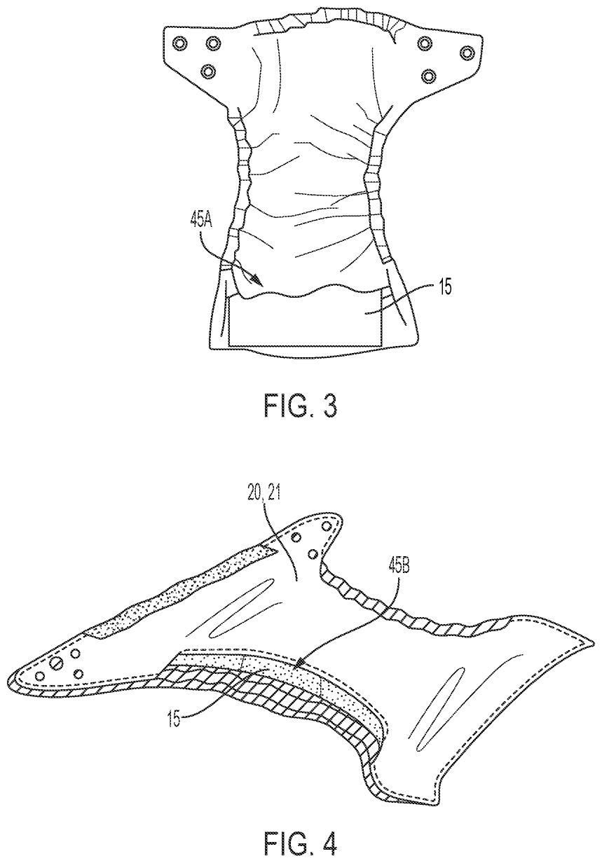

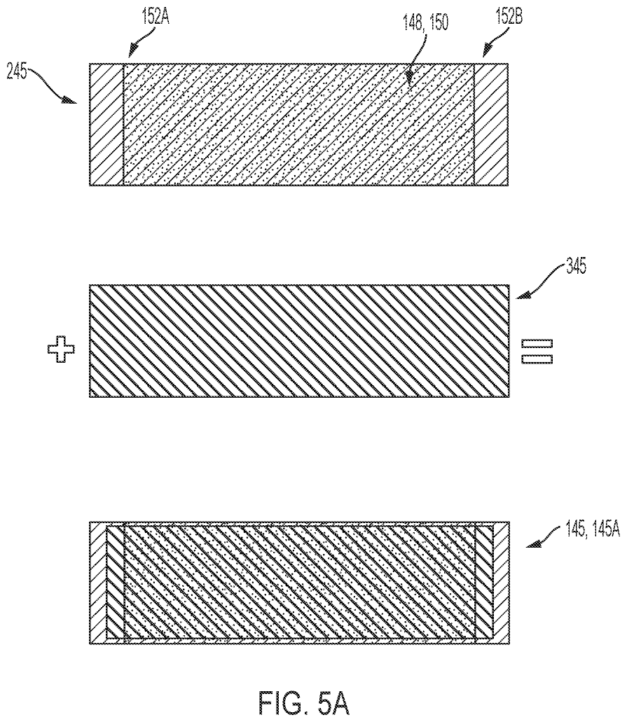

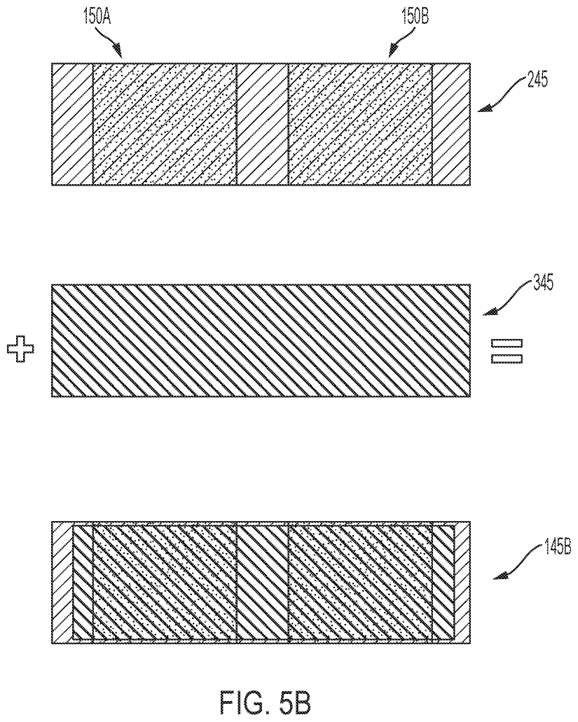

Referring again to FIG. 1, an interior portion facing toward a wearer may include an opening 13 that defines an interior pocket 45A (FIG. 3) to receive a reusable absorbent (carrier) insert 15 (FIG. 3) (also depicted as reusable absorbent carrier inserts 245 in FIGS. 5A-10). In the in-use configuration, the outer shell 21 may at least partially enclose the reusable absorbent insert 15 as shown in FIGS. 3-4, forming a pocket 45 in which the absorbent insert resides during use. In embodiments, the outer shell 21 may include a single layer including an inner, wearer-facing side on top of which the reusable absorbent inert 15 is positioned. As a non-limiting example, the reusable absorbent insert in FIG. 3 is shown partially disposed in the interior pocket 45A defined at an end in the front portion 11 of the outer shell of the absorbent assembly. The interior pocket 45A may be defined any location within the inner panel, including and not limited to, for example, an end in the back portion 12, along side edges (as shown by interior pocket 45B of FIG. 4), in central portions, or at other portions of the inner panel within which the reusable absorbent insert 15 may be received. In embodiments, the inner panel may be a fleece material made of polyester or like materials. As described in greater detail further below, the reusable absorbent insert 15 may be made of multiple layers of cotton or reusable pads.

FIG. 2 shows an exterior portion of the outer shell that faces away from the wearer. Fasteners 123 and 124 of elongated wings 126 and 127 (FIG. 1) of the back portion 12 are configured to fasten to fasteners 112, 113, and 114 of the front portion 11 when the outer shell is in a folded, fastened configuration for the wearer. In embodiments, the fasteners may be snaps or other suitable fastening mechanisms. Nonlimiting examples of engageable fastening and receiving components include tape tabs, hook and loop fastening components, interlocking fasteners such as tabs & slots, buckles, buttons, snaps, and/or hermaphroditic fastening components. Some exemplary surface fastening systems are disclosed in U.S. Pat. Nos. 3,848,594; 4,662,875; 4,846,815; 4,894,060; 4,946,527; 5,151,092; and 5,221,274.

As noted above, in the in-use configuration of the present invention, the outer shell 21 at least partially encloses the absorbent insert 15, forming a pocket 45 in which the absorbent insert resides during use. The wearer-facing surface of the outer shell 21 may cover at least about 60%, or at least about 75% or at least about 80%, or at least about 90%, or from about 60% to about 100% of the surface area of the absorbent insert 15, when the absorbent insert 15 is fully positioned within the pocket 45 as designed, reciting for said range every 5% increment therein. In some nonlimiting examples, the width of the outer shell 21 wearer-facing surface is greater than the width of the absorbent insert 15 throughout a majority of, or all of, the length of the absorbent insert. Additionally, or alternatively, the garment-facing surface of the outer shell 21 may cover at least about 60%, or at least about 75% or at least about 80%, or at least about 90%, or from about 60% to about 100% of the surface area of the absorbent insert 15, when the absorbent insert 15 is fully positioned within the pocket 45 as designed, reciting for said range every 5% increment therein. In some nonlimiting examples, the width of the outer shell 21 garment-facing surface is greater than the width of the absorbent insert 15 throughout a majority of, or all of, the length of the absorbent insert 15.

The outer shell 21 comprises one or more openings 13 which are capable of receiving an absorbent insert 15, as shown in FIGS. 1 and 3-4. The openings 13 are sized and shaped to permit the absorbent insert 15 to be inserted into the pocket 45 by the user. In various embodiments, an opening 13 comprises a maximum longitudinal length, Lo, that is at least about 50%, or at least about 60%, or at least about 70%, or from about 50% to about 100%, or from about 55% to about 85%, or from about 60% to about 75% of the length of the outer shell 21, Lc, reciting for each range every 5% increment therein.

The one or more openings 13 may include at least one longitudinally-extending opening defining a pocket such as interior pocket 45B (FIG. 4). By longitudinally-extending, it is meant that the opening extends more in the longitudinal dimension than in the lateral dimension. For purposes of longitudinally-extending, the longitudinal direction includes directions within 40 degrees of the maximum linear dimension in the x-y plane of the outer shell 21. The longitudinally-extending opening may be present in the crotch region. The longitudinally-extending opening may extend into one or both of the front and rear regions.

The opening 13 may be sized and shaped such that the absorbent insert 15 will remain in the interior pocket 45 once inserted. For example, the opening 13 may be smaller in one or more dimensions than the insert 15. Additionally, or alternatively, a second edge of the opening 13 may be spaced from the a edge of the opening 13 such that less than 10 mm of void space in the z-direction exists above or below the absorbent insert 15 within the pocket 45.

The opening 13 may be sealable, such that it may be closed or repeatedly opened and reclosed. In nonlimiting examples, the first edge and/or the second edge may include means for attaching to each other, including any of the fastening components and/or receiving components described above, as well as pressure sensitive adhesive. Additionally, or alternatively, the first or second edge may be capable of wrapping about the longitudinal edge and being secured to an exterior surface.

Referring again to FIGS. 3-4, the reusable absorbent insert 15 may be designed to contain and/or absorb body exudates, and may be made of pliable materials as will be described further below. The reusable absorbent insert 15 may be made of washable material such as cloth, or of a material suitable for composting, such as cellulose. In other embodiments, the absorbent insert may be disposable an made from materials commonly found in disposable absorbent articles. The reusable absorbent insert 15 may have a topsheet and a backsheet that may be joined together. An absorbent core may be disposed between the topsheet and the backsheet.

The topsheet is generally a portion of the insert 15 that may be positioned at least in partial contact or close proximity to a wearer. Suitable topsheets may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet is generally supple, soft feeling, and non-irritating to a wearer's skin. Generally, at least a portion of the topsheet is liquid pervious, permitting liquid to readily penetrate through the thickness of the topsheet. The topsheet may comprise one or more apertures.

Any portion of the topsheet may be coated with a lotion, antibacterial or skin care composition as is known in the art. The topsheet may be fully or partially elasticized or may be foreshortened so as to provide a void space between the topsheet and the core. Topsheet, backsheet or any portion of the topsheet or backsheet may be embossed and/or matte finished to provide a more cloth-like appearance.

The backsheet is generally the outer liner portion of insert 15 forming the garment-facing surface thereof, and prevents the exudates absorbed and contained within insert 15 from wicking through and soiling the outer shell 21. The backsheet may comprise one or more nonwovens, films, elastomeric films, foams, strands, or combinations of these or other suitable materials with nonwovens or synthetic films. In nonlimiting examples, the backsheet is a laminate of an elastomeric material, such as a film, and a nonwoven.

In various embodiments, the backsheet is substantially water-impermeable. Suitable backsheet materials include nonwovens and/or films. Other suitable backsheet materials may include breathable materials that permit vapors to escape from the insert 15 while still preventing exudates from passing through the backsheet. Exemplary breathable materials may include materials such as woven webs, nonwoven webs, composite materials such as film-coated nonwoven webs, and microporous films.

Other suitable materials and/or manufacturing techniques may be used to provide a suitable backsheet including, but not limited to, surface treatments, particular film selections and processing, particular filament selections and processing. Backsheet may be joined to topsheet, absorbent core or any other element of insert 15 by any suitable attachment mechanism known in the art. For example, the attachment mechanism may include a continuous line or layer of adhesive, a patterned layer of adhesive, or an array of separate lines, spirals, or spots of adhesive.

As described above, the insert 15 may have an absorbent core disposed within an envelope-like structure formed by the topsheet and backsheet. The absorbent core may comprise materials suitable for absorbency and for washing and reuse of the insert. In nonlimiting examples, the insert 15 may be formed of cotton, fleece, flannel, felt, nylon, polyester, terry cloth and combinations thereof.

FIGS. 5A-10 show various embodiments of the reusable absorbent (carrier) insert 15 as described herein as reusable absorbent carrier insert 245 in combination with disposable insert 345 to form reusable absorbent carrier insert and disposable insert combinations 145. As described above, the reusable absorbent carrier insert 15, 245 (and thus the reusable absorbent carrier insert and disposable insert combinations 145) is configured to be inserted into an interior pocket 45, 45A, 45B of an outer shell 21 of an absorbent assembly 20.

Thus, in the embodiments herein and as will be described in greater detail below, the reusable absorbent carrier insert and disposable insert combinations 145 of FIGS. 5A-10 include a reusable absorbent carrier insert 245 and a disposable insert 345. The reusable absorbent carrier insert 245 includes a carrier portion 148, a first length measured along a central longitudinal axis of the reusable absorbent carrier insert 245, and a first width measured along a central lateral axis of the reusable absorbent carrier insert 245. The reusable absorbent carrier insert 245 further has a first capacity-to-caliper ratio. The carrier portion 148 of the reusable absorbent carrier insert 245 may include elastomeric material. The carrier portion 148 of the reusable absorbent carrier insert 245 may be attached to the reusable absorbent carrier insert 245 via a fastening mechanism comprising an overstitch, a flat seam, an adhesive, or combinations thereof. The carrier portion 148 of the reusable absorbent carrier insert 245 prior to receiving the disposable insert 345 may include an exterior portion that is exterior facing and an interior portion that is interior facing.

The disposable insert 345 includes a superabsorbent material, a second length measured along a central longitudinal axis of the disposable insert 345, and a second width measured along a central lateral axis of the disposable insert 345. The disposable insert 345 has a second capacity-to-caliper ratio. The reusable absorbent carrier insert 245 is configured to receive and retain the disposable insert 345 such that the disposable insert 345 is configured to be received and contained within the carrier portion 148 of the reusable absorbent carrier insert 245 to form the reusable absorbent carrier insert and disposable insert combination 145. The superabsorbent material may include an absorbent gelling materials (AGM), and the reusable absorbent carrier insert may include one or more textile fabrics and not be an AGM.

The first length is greater than the second length, the first width is greater than the second width, and the first capacity-to-caliper ratio of the reusable absorbent carrier insert 245 is less than the second capacity-to-caliper ratio of the disposable insert 345 such that the disposable insert 345 having the second capacity-to-caliper ratio that is greater than the first capacity-to-caliper ratio of the reusable absorbent carrier insert 245 is thinner than the reusable absorbent carrier insert 245. A caliper defines a thickness of a material when dry, and a capacity defines a body fluid storage capacity. In embodiments, the reusable absorbent carrier insert and disposable insert combinations 145 as described herein are thinner and have a greater capacity to caliper ratio than a reusable absorbent insert alone that has a substantially same capacity. At least a portion of the reusable absorbent carrier insert 245 may be disposed between the disposable insert 345 and a skin surface of a wearer, when the reusable absorbent carrier insert and disposable insert combination 145 is in a wear configuration.

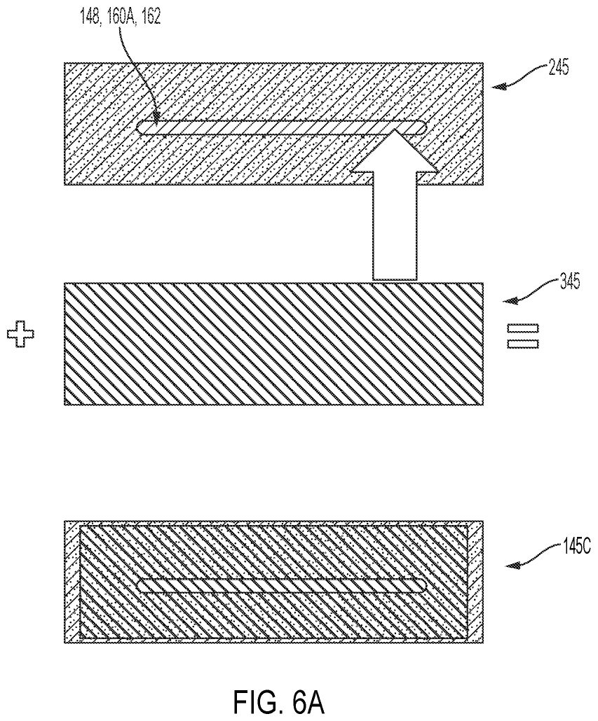

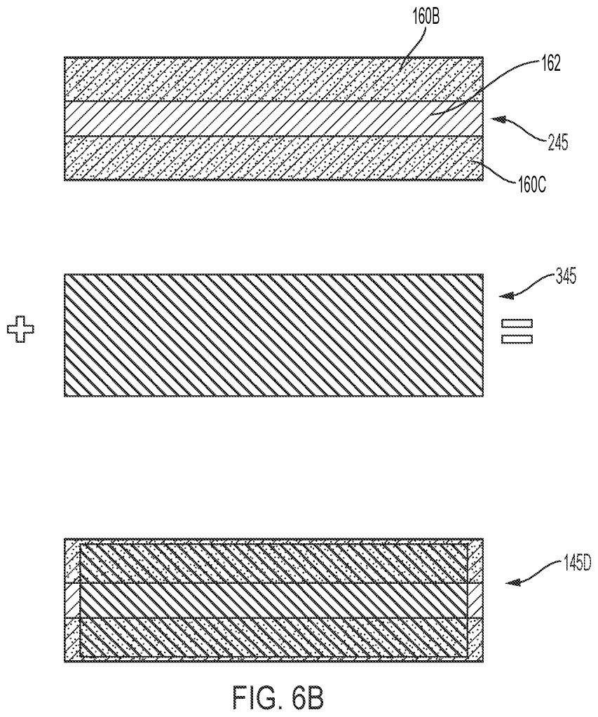

In embodiments, the reusable absorbent carrier insert 245 is configured to be inserted into an interior pocket 45 of an outer shell 21 of an absorbent pant 20. Additionally or alternatively, as shown within the embodiments of FIGS. 5A-10, which are each described in greater detail further below, the carrier portion 148 of the reusable absorbent carrier insert 245 may include at least a belt 150 with two open ends 152A, 152B, at least a pouch 160, or combinations thereof. As shown within the embodiments of FIGS. 6A-10, the pouch 160 may include a longitudinal incision or a diagonal incision. As shown within the embodiments of FIGS. 6A-8 and 10, the longitudinal incision extends at least partially across the reusable absorbent carrier insert 245. As shown within the embodiment of FIG. 9, the diagonal incision extends at least partially across the reusable absorbent carrier insert 245. A method of forming a reusable absorbent carrier insert and disposable insert combinations 145 described herein may include receiving the disposable insert 345 comprising the superabsorbent material within the carrier portion 148 of the reusable absorbent carrier insert 245, and containing the disposable insert 345 in the carrier portion 148 in a self-locking configuration upon receipt.

FIG. 5A depicts a reusable absorbent carrier insert and disposable insert combination 145A, in which a reusable absorbent carrier insert 245 including a belt 150 with two open ends 152A, 152B positioned on opposite ends of the belt 150. The reusable absorbent carrier insert 245 is configured to receive and retain the disposable insert 345 within the belt 150. The disposable insert 345 is configured to be received within the interior portion to form the reusable absorbent carrier insert and disposable insert combination 145A. The carrier portion 148 of the reusable absorbent carrier insert 245 comprises at least the belt 150 with two open ends 152A, 152B, and the disposable insert 345 is configured to be received within the interior portion of the at least the belt 150.

FIG. 5B depicts a reusable absorbent carrier insert and disposable insert combination 145B including a reusable absorbent carrier insert 245 having a pair of belts 150A, 150B. The belts 150A, 150B each include two open ends. The disposable insert 345 is configured to be received within the interior portion to form the reusable absorbent carrier insert and disposable insert combination 145B. The carrier portion 148 of the reusable absorbent carrier insert 245 comprises at least the belts 150A, 150B with two open ends each, and the disposable insert 345 is configured to be received within the interior portion of the at least the belts 150A, 150B.

FIG. 6A depicts a reusable absorbent carrier insert and disposable insert combination 145C including a reusable absorbent carrier insert 245 having a pouch 160A. The pouch 160A includes a partially extending longitudinal incision 162. The disposable insert 345 is configured to be received within the interior portion to form the reusable absorbent carrier insert and disposable insert combination 145C. The carrier portion 148 of the reusable absorbent carrier insert comprises at least the pouch 160A, and the disposable insert 345 is configured to be received within the interior portion of the at least the pouch 160A.

FIG. 6B depicts a reusable absorbent carrier insert and disposable insert combination 145D including a reusable absorbent carrier insert 245 having a pouch 160 with a fully extending longitudinal incision 162. The fully extending longitudinal incision 162 is disposed between end portions of the pouch 160B, 160C. The disposable insert 345 is configured to be received within the interior portion to form the reusable absorbent carrier insert and disposable insert combination 145D. The carrier portion 148 of the reusable absorbent carrier insert comprises at least the pouch 160, and the disposable insert 345 is configured to be received within the interior portion of the at least the pouch 160, such as between end portions of the pouch 160B, 160C.

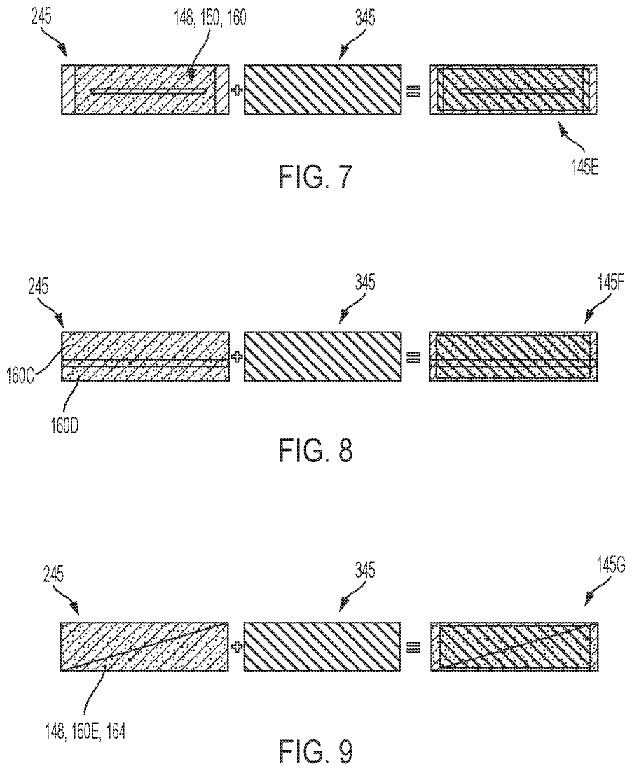

FIG. 7 depicts a reusable absorbent carrier insert and disposable insert combination 145E including a reusable absorbent carrier insert 245 having a belt 150 and a pouch 160 as part of the carrier portion 148. The belt 150 includes two open ends. The pouch 160 includes a partially extending longitudinal incision. The disposable insert 345 is configured to be received within the interior portion to form the reusable absorbent carrier insert and disposable insert combination 145E. The carrier portion 148 of the reusable absorbent carrier insert 245 comprises at least the belt 150 with two open ends, and the disposable insert 345 is configured to be received within the interior portion of the at least the belt 150. The carrier portion 148 of the reusable absorbent carrier insert comprises at least the pouch 160, and the disposable insert 345 is configured to be received within the interior portion of the at least the pouch 160. In the embodiment of FIG. 7, the carrier portion 148 of the reusable absorbent carrier insert comprises at least the belt 150 with two open ends and the pouch 160, and the disposable insert 345 is configured to be received within the interior portion of the at least the belt 160.

FIG. 8 depicts a reusable absorbent carrier insert and disposable insert combination 145F including a reusable absorbent carrier insert 245 having a pouch 160. The pouch 160 includes a pair of flaps 160C, 160D. The disposable insert 345 is configured to be received within the interior portion to form the reusable absorbent carrier insert and disposable insert combination 145F. The carrier portion 148 of the reusable absorbent carrier insert comprises at least the pouch 160, and the disposable insert 345 is configured to be received within the interior portion of the at least the pouch 160, such as between the pair of flaps 160C. 160D. In the embodiment of FIG. 8, the carrier portion 148 of the reusable absorbent carrier insert 245 comprises at least a first flap 160C and a second flap 160D. The first flat 160C is longer than the second flap 160D in a vertical axis direction and configured to overlap the second flap 160D. The disposable insert 345 is configured to be received within the interior portion of the second flap 160D and covered by the first flap 160C.

FIG. 9 depicts a reusable absorbent carrier insert and disposable insert combination 145G including a reusable absorbent carrier insert 245 having a pouch 160E. The pouch 160E includes a diagonal incision 164. The disposable insert 345 is configured to be received within the interior portion to form the reusable absorbent carrier insert and disposable insert combination 145G. The carrier portion 148 of the reusable absorbent carrier insert comprises at least the pouch 160E, and the disposable insert 345 is configured to be received within the interior portion of the at least the pouch 160E.

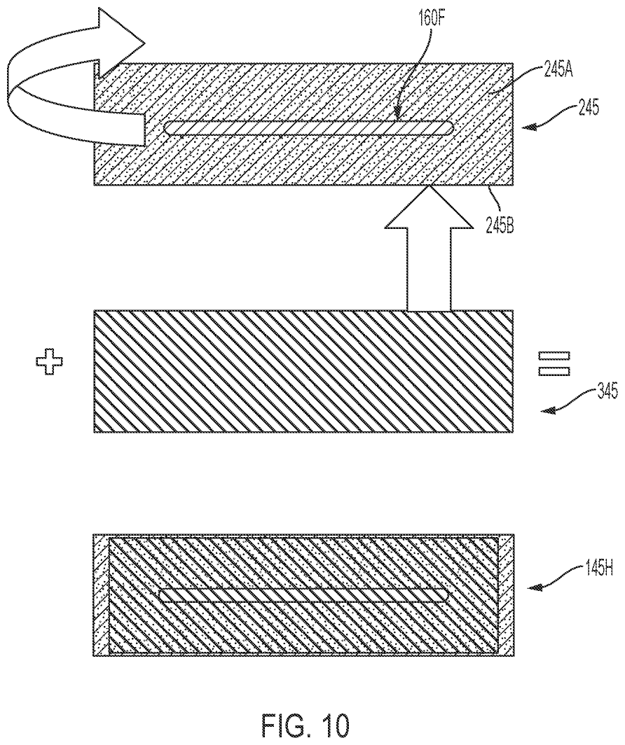

FIG. 10 depicts a reusable absorbent carrier insert and disposable insert combination 145H including a reusable absorbent carrier insert 245 having a pouch 160F. The pouch 160F includes a partially extending longitudinal incision that is configured to be folded over the disposable insert 345. Thus, when the reusable absorbent carrier insert and disposable insert combination 145H is formed, the interior portion is folded over the disposable insert and is exterior facing. The carrier portion 148 of the reusable absorbent carrier insert comprises at least the pouch 160F, and the disposable insert 345 is configured to be received within the interior portion of the at least the pouch 160F when folded over the disposable insert 345.

Embodiments of the reusable absorbent carrier insert and disposable insert combinations 145 described herein may allow for a longer usage time as compared to a reusable absorbent insert alone in an absorbent article such as a diaper, while allowing for the durability and reusability, and without increasing dry caliper (thickness). The disposable insert may increase the absorbency capacity of the insert combination without significantly increasing dry caliper (thickness) of the insert combination. The increase in usage time is thus able to be reached while decreasing the caliper in dry condition and maintaining a typical caliper of a reusable absorbent insert in wet condition at comparable capacities. Further, the reusable absorbent carrier insert and disposable insert combinations 145 allowed for a thinner cloth for the reusable absorbent carrier insert to be used compared to the reusable absorbent insert alone having similar capacity, allowing for comfortable fit, less bulk, and faster drying and overall efficient dryness against the skin (such as via the fluid locked away in the AGM of the disposable insert) and increased mileage (e.g., longer usage time). The reusable absorbent carrier insert and disposable insert combinations 145 may thus have a larger capacity-to-caliper ratio than the reusable absorbent insert alone having similar capacity. The capacity-to-caliper ratio and the combined insert weight-to-capacity may be expressed in metrics of “ml capacity” per “mm caliper” or “ml capacity” per “g.”

In embodiments, the calipers mentioned may be at 2.1 kPa (0.3 PSI) pressure during measurement. An AGM laminate may have a caliper of approximately 1.5 mm, at 435 g/m² AGM, with a CRC (Centrifuge Retention Capacity [g_{Absorbed}/g_{AGM}]) of 1.22+/−0.5 g/cm² and an AAP (Absorbency against Pressure @ 0.7 psi [g_{Absorbed}/g_{AGM}]) capacity of 1.09+/−0.5 g/cm². Such a laminate can reduce rewet (Collagen Rewet at 3.4 psi) versus a referenced cloth insert to 50% of initial value and increase capacity. The capacity can easily be raised within the limits of the technology at moderate dry caliper increases (such as a caliper of ≤4 mm). In embodiments, a very high CRC (>30 g/g) and/or very low SFC (0-5) AGM at high FSR (Free Swell Rate [g_{Absorbed}/g_{AGM}×s_{Absorb. Time}]) may be found. Regarding AGM fibers, such fibers may have (i.e., for needle punched pure AGM fiber) up to 100% absorbent material in the disposable insert, at various AGM fiber properties. Typically, for a 400 g/m² AGM-fiber core, the caliper may be at 4-5 mm. For foam inserts, the caliper may be at 2 mm, with two or more layers utilized to arrive at a targeted Rewet reduction of 50% of an initial cloth insert rewet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A reusable absorbent carrier insert and disposable insert combination, comprising:
a reusable absorbent carrier insert, the reusable absorbent carrier insert comprising a carrier portion, a first length measured along a central longitudinal axis of the reusable absorbent carrier insert, and a first width measured along a central lateral axis of the reusable absorbent carrier insert, wherein the reusable absorbent carrier insert has a first capacity-to-caliper ratio; and
a disposable insert, the disposable insert comprising an absorbent material, a second length measured along a central longitudinal axis of the disposable insert, and a second width measured along a central lateral axis of the disposable insert, wherein the disposable insert has a second capacity-to-caliper ratio;
wherein the reusable absorbent carrier insert is configured to receive and retain the disposable insert;
wherein the disposable insert is configured to be received and contained within the carrier portion of the reusable absorbent carrier insert to form the reusable absorbent carrier insert and disposable insert combination;
wherein the first length is greater than the second length;
wherein the first width is greater than the second width; and
wherein the first capacity-to-caliper ratio is less than the second capacity-to-caliper ratio.

2. The reusable absorbent carrier insert and disposable insert combination of claim 1, wherein the reusable absorbent carrier insert is configured to be inserted into an interior pocket of an outer shell of an absorbent pant, on top an interior portion of the outer shell, or combinations thereof.

3. The reusable absorbent carrier insert and disposable insert combination of claim 1, wherein the carrier portion of the reusable absorbent carrier insert comprises elastomeric material.

4. The reusable absorbent carrier insert and disposable insert combination of claim 1, wherein the carrier portion of the reusable absorbent carrier insert comprises at least a belt with two open ends, at least a pouch, or combinations thereof, wherein the at least a pouch comprises a longitudinal incision or a diagonal incision.

5. The reusable absorbent carrier insert and disposable insert combination of claim 4, wherein the longitudinal incision extends at least partially across the reusable absorbent carrier insert.

6. The reusable absorbent carrier insert and disposable insert combination of claim 4, wherein the diagonal incision extends at least partially across the reusable absorbent carrier insert.

7. The reusable absorbent carrier insert and disposable insert combination of claim 1, wherein the carrier portion of the reusable absorbent carrier insert is attached to the reusable absorbent carrier insert via a fastening mechanism comprising an overstitch, a flat seam, an adhesive, or combinations thereof.

8. The reusable absorbent carrier insert and disposable insert combination of claim 1, wherein the carrier portion of the reusable absorbent carrier insert prior to receiving the disposable insert comprises an exterior portion that is exterior facing and an interior portion that is interior facing.

9. The reusable absorbent carrier insert and disposable insert combination of claim 8, wherein, when the reusable absorbent carrier insert and disposable insert combination is formed, the interior portion is folded over the disposable insert and is exterior facing.

10. The reusable absorbent carrier insert and disposable insert combination of claim 8, wherein the disposable insert is configured to be received within the interior portion to form the reusable absorbent carrier insert and disposable insert combination.

11. The reusable absorbent carrier insert and disposable insert combination of claim 10, wherein the carrier portion of the reusable absorbent carrier insert comprises at least a belt with two open ends, and the disposable insert is configured to be received within the interior portion of the at least a belt.

12. The reusable absorbent carrier insert and disposable insert combination of claim 10, wherein the carrier portion of the reusable absorbent carrier insert comprises at least a pouch, and the disposable insert is configured to be received within the interior portion of the at least a pouch.

13. The reusable absorbent carrier insert and disposable insert combination of claim 10, wherein the carrier portion of the reusable absorbent carrier insert comprises at least a belt with two open ends and a pouch, and the disposable insert is configured to be received within the interior portion of the at least a belt.

14. The reusable absorbent carrier insert and disposable insert combination of claim 10, wherein the carrier portion of the reusable absorbent carrier insert comprises at least a first flap and a second flap, the first flat longer than the second flap and configured to overlap the second flap, and the disposable insert is configured to be received within the interior portion of the second flap and covered by the first flap.

15. The reusable absorbent carrier insert and disposable insert combination of claim 1, wherein the absorbent material comprises an absorbent gelling materials (AGM), and the reusable absorbent carrier insert comprises one or more textile fabrics and does not comprise AGM.

16. The reusable absorbent carrier insert and disposable insert combination of claim 1, wherein at least a portion of the reusable absorbent carrier insert is disposed between the disposable insert and a skin surface of a wearer, when the reusable absorbent carrier insert and disposable insert combination is in a wear configuration.

17. An absorbent pant, comprising:
an outer shell comprising an interior pocket, an interior portion, or combinations thereof; and
a reusable absorbent carrier insert and disposable insert combination configured to be received at least one of in the interior pocket of the outer shell of the absorbent pant, on top of the interior portion of the outer shell, or combinations thereof, the reusable absorbent carrier insert and disposable insert combination comprising:
a reusable absorbent carrier insert, the reusable absorbent carrier insert comprising a carrier portion, a first length measured along a central longitudinal axis of the reusable absorbent carrier insert, and a first width measured along a central lateral axis of the reusable absorbent carrier insert, wherein the reusable absorbent carrier insert has a first capacity-to-caliper ratio; and
a disposable insert, the disposable insert comprising an absorbent material, a second length measured along a central longitudinal axis of the disposable insert, and a second width measured along a central lateral axis of the disposable insert, wherein the disposable insert has a second capacity-to-caliper ratio;
wherein the reusable absorbent carrier insert is configured to receive and retain the disposable insert;
wherein the disposable insert is configured to be received and contained within the carrier portion of the reusable absorbent carrier insert to form the reusable absorbent carrier insert and disposable insert combination;
wherein the first length is greater than the second length;
wherein the first width is greater than the second width; and
wherein the first capacity-to-caliper ratio is less than the second capacity-to-caliper ratio.

18. The absorbent pant of claim 17, wherein the carrier portion comprises an exterior portion that is exterior facing and an interior portion that is interior facing prior to receipt of the disposable insert.

19. The absorbent pant of claim 18, wherein, when the reusable absorbent carrier insert and disposable insert combination is formed, the interior portion is folded over the disposable insert and is exterior facing.

20. The absorbent pant of claim 18, wherein, when the reusable absorbent carrier insert and disposable insert combination is formed, the disposable insert is received into the interior portion that remains interior facing.

21. A method of forming a reusable absorbent carrier insert and disposable insert combination, the method comprising:
receiving a disposable insert comprising an absorbent material within a carrier portion of a reusable absorbent carrier insert; and
containing the disposable insert in the carrier portion in a self-locking configuration upon receipt;
wherein the reusable absorbent carrier insert has a first capacity-to-caliper ratio, wherein the disposable insert has a second capacity-to-caliper ratio, and wherein the first capacity-to-caliper ratio is less than the second capacity-to-caliper ratio.
